(19) United States
(12) Patent Application Publication
(10) Pub. No.: US 2025/0375185 A1
(43) Pub. Date: Dec. 11, 2025

(54) CMUT MEDICAL DEVICES, FABRICATION METHODS, SYSTEMS, AND RELATED METHODS

(71) Applicant: XENTER, INC., Draper, UT (US)

(72) Inventors: Liu JIANWEI, Draper, UT (US); F. Levent DEGERTEKIN, Draper, UT (US); Sarp SATIR, Draper, UT (US); Richard J. LINDER, Draper, UT (US)

(21) Appl. No.: 18/866,149

(22) PCT Filed: May 16, 2023
(86) PCT No.: PCT/US2023/022337

§ 371 Date: Nov. 15, 2024

Related U.S. Application Data
(60) Provisional application No. 63/342,527, filed on May 16, 2022.
(60) Provisional application No. 63/342,812, filed on May 17, 2022.

(51) Int. Cl. A61B 8/00; A61B 8/08; A61B 8/12; B06B 1/02; G01N 29/24

(52) U.S. Cl. CPC A61B 8/4444; A61B 8/085; A61B 8/12; A61B 8/4477; A61B 8/56; B06B 1/0292; G01N 29/2406; A61B 2562/0247; B06B 2201/76

(57) ABSTRACT

Capacitive Micromachined Ultrasonic Transducer (CMUT) device, methods of manufacture, and systems and methods of use. The CMUTs have a substrate, peripheral walls defining a boundary of a cavity, and a membrane bonded to the peripheral walls. Protruding from the substrate are posts that are enclosed by the peripheral walls. The posts contact, but are not bonded to, the membrane, forming a cavity that resonates with certain acoustic frequencies. The CMUTs are placed into a vascular system of a patient in a stent, a catheter, or other small device. The CMUTs are responsive to acoustic frequencies emitted from a device outside the body, thereby acting as sensors for various characteristics of the environment around the CMUT inside the body.

### CROSS-REFERENCE TO RELATED APPLICATIONS

This application claims priority to U.S. Provisional Patent Application No. 63/342,527 filed on May 16, 2022, and entitled CMUT DEVICE AND FABRICATION METHOD, and to U.S. Provisional Patent Application No. 63/342,812 filed on May 17, 2022 and entitled MEDICAL DEVICES, SYSTEMS AND RELATED METHODS, both of which are incorporated herein by reference in their entireties.

### BACKGROUND

The present invention relates generally to medical devices incorporating sensors and electronics. In one, non-limiting example, such medical devices may include intraluminal devices, such as guidewires and catheters, which include various sensors for imaging and/or measuring of one or more physiological parameters. In other non-limiting examples, medical devices may include implantable sensors to provide imaging, monitor or measure physiological parameters.

Among these medical devices are Capacitive Micromachined Ultrasonic Transducers (CMUTs) that are a relatively new technology within the field of transducers. In many applications CMUTs can be used to generate ultrasonic waves and/or receive ultrasonic waves. Due to their small size, CMUTs provide unique benefits within medical devices that require tight tolerances and small sensors. Fabrication of such devices can be very challenging when considering issues of performance, yield, reliability, costs, etc.

There is an ongoing need for improved medical devices that effectively integrate sensors and can help provide data in a more efficient manner and/or provide data not previously obtainable in a practical manner.

The subject matter claimed herein is not limited to embodiments that solve any disadvantages or that operate only in environments such as those described above. Rather, this background is only provided to illustrate one exemplary technology area where some embodiments described herein may be practiced.

### SUMMARY

At least one embodiment disclosed herein comprises a capacitive micromachined ultrasonic transducer (CMUT) apparatus comprising a substrate, which may include a CMOS (complementary metal-oxide silicon) wafer, with one or more peripheral walls protruding from the substrate. The one or more peripheral walls define an outer boundary of a cavity. Additionally, a silicon-on-insulator (SOI) wafer with a highly doped silicon layer on top may be bonded to the one or more peripheral walls such that the cavity is positioned between the SOI and the substrate. A membrane positioned on the side of the cavity may be formed upon removal of a handling wafer from the SOI wafer.

The CMUT may also comprise one or more posts protruding from the substrate. The one or more posts may be enclosed by the one or more peripheral walls. At least a portion of the one or more posts comprise a width of less than 10 microns. Additionally, one or more interior walls may protrude from the substrate. The one or more interior walls may be enclosed by the one or more peripheral walls.

Additionally, at least one embodiment may include a capacitive micromachined ultrasonic transducer (CMUT) apparatus. The apparatus includes a substrate, one or more peripheral walls protruding from the substrate, the one or more peripheral walls defining an outer boundary of a cavity, and a membrane bonded to the one or more peripheral walls, the cavity being positioned between the membrane and the substrate. The apparatus also includes one or more posts protruding from the substrate, the one or more posts enclosed by the one or more peripheral walls, wherein at least a portion of the one or more posts comprise a width of less than 10 microns. The apparatus also has one or more interior walls protruding from the substrate, the one or more interior walls enclosed by the one or more peripheral walls.

Further embodiments of the present disclosure are directed to a method for constructing a capacitive micromachined ultrasonic transducer (CMUT). The method includes disposing, on a substrate, one or more peripheral walls, the one or more peripheral walls defining an outer boundary of a cavity, wherein the one or more peripheral walls comprise a width of at least 8 microns. The method continues by disposing, on the substrate, one or more posts, the one or more posts enclosed by the one or more peripheral walls, wherein at least a portion of the one or more posts comprise a width of 1 micron to 10 microns. The method also includes disposing, on the substrate, one or more interior walls protruding from the substrate, the one or more interior walls enclosed by the one or more peripheral walls, wherein at least a portion of the one or more interior walls comprise a width of 0.5 microns to 1 micron.

Further embodiments of the present disclosure are directed to a medical device having a body and at least one electronic component associated with the body. The at least one electronic component includes at least one sensor and an ultrasonic transducer configured to receive ultrasonic signals and thereby power the at least one sensor. The electronic component is configured to provide a response ultrasonic wave responsive to a determination by the at least one sensor.

Still further embodiments of the present disclosure are directed to a system including an external device having a first ultrasonic transducer, a medical device configured to be positioned within a patient's anatomy. The medical device includes at least one electronic component, at least one sensor, and a second ultrasonic transducer configured to receive ultrasonic signals generated by the first ultrasonic transducer to power the at least one sensor. The electronic component is configured to provide a response ultrasonic wave to the external device responsive to a determination by the at least one sensor.

Yet other embodiments of the present disclosure are directed to a method including implanting an electronic component within a patient adjacent to, or within, a tumor, powering the electronic component using ultrasonic energy, and detecting a water content of the tumor using a sensor of the electronic component. The method also includes providing a response signal from the electronic component to an external device based on the detected water content, and determining a density of, or a change in density of, the tumor based on the response signal.

Other embodiments of the present disclosure are directed to a medical device including a flexible, elongated member having a distal portion configured for insertion into a vessel of a patient, and an imaging device disposed in the distal portion of the elongated member. The imaging device includes a flexible substrate, a plurality of ultrasonic transducers arranged on the flexible substrate, each of the plurality of ultrasonic transducers comprising a capacitive micromachined ultrasonic transducer on complementary metal oxide silicon (CMUT on CMOS) device, and a tail extending from the flexible substrate having a plurality of connection pads.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claim or may be learned by the practice of the invention as set forth hereinafter.

### BRIEF DESCRIPTION OF THE SEVERAL VIEWS OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings described below.

FIG. 1 illustrates a side view of a silicon-on-insulator (SOI) wafer according to embodiments of the present disclosure.

FIG. 2 illustrates a side view of a complementary metal-oxide semiconductor (CMOS) wafer with lower electrodes according to embodiments of the present disclosure.

FIG. 3 illustrates a side view of a CMOS wafer cavity with oxide layers according to embodiments of the present disclosure.

FIG. 4 illustrates a side view of a CMOS wafer bonding according to embodiments of the present disclosure.

FIG. 5 illustrates a side view of a CMUT according to embodiments of the present disclosure.

FIG. 6 illustrates a top view of a CMOS wafer without a membrane according to embodiments of the present disclosure.

FIG. 7 depicts a flowchart of steps in a method for fabricating a CMUT according to embodiments of the present disclosure.

FIG. 8 illustrates a catheter according to embodiments of the present disclosure.

FIG. 9 is an enlarged view of a portion of the catheter depicted in FIG. 8.

FIG. 10 is a cross-sectional view along the lines 10-10 as shown in FIG. 9.

FIG. 11 depicts a sensor array that may be utilized in the catheter shown in FIG. 8.

FIG. 12 illustrates a stent according to embodiments of the present disclosure.

FIGS. 13 and 14 illustrate a system according to embodiments of the present disclosure.

FIG. 15 illustrates another system according to embodiments of the present disclosure.

FIG. 16 illustrates a transaortic valve according to embodiments of the present disclosure.

FIG. 17 depicts another system according to embodiments of the present disclosure.

FIG. 18 illustrates a wearable patch according to embodiments of the present disclosure.

### DETAILED DESCRIPTION OF THE INVENTION

The present disclosure provides various examples and embodiments of medical devices, systems, methods, and related components. Such devices, systems, etc., may be used in the diagnosis and/or treatment of physical conditions including the detection or determination of desired parameters or characteristics. Disclosed embodiments include capacitive micromachined ultrasonic transducers (CMUTs). In some embodiments, the CMUTs are useable in medical devices. For example, disclosed CMUTs may be used in implantable sensors, IVUS catheters, ultrasonic imaging devices, and/or other medical devices that utilize transducers. The transducers can be used to induce ultrasonic signals and/or to receive ultrasonic signals.

Conventional CMUTs comprise a top electrode that is integrated within a membrane, a bottom substrate, and a cavity formed between the membrane and the substrate. As a force is applied to the conductive membrane, the membrane deforms. The deformity of the membrane creates a variation in the capacitance between the membrane and the substrate. The resulting change in the electric field can be detected and/or produced by circuitry integrated into the CMUT. Conversely, application of an electrical signal to the substrate (e.g., an electrode below the membrane) causes the membrane to deform, resulting in the production of acoustic waves by the vibrating membrane. Conversely, a change in capacitance between the membrane and the substrate can create a displacement of the membrane, creating ultrasonic waves.

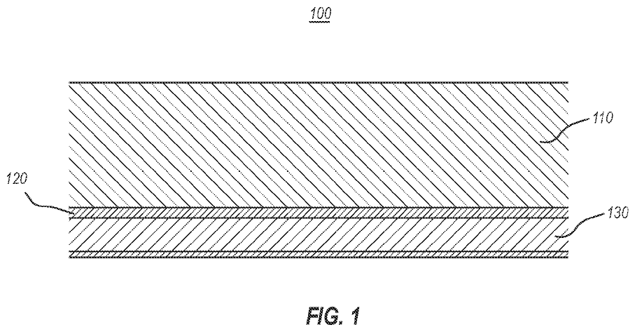

CMUT fabrication may occur through a variety of different methods, including the use of a sacrificial layer, cavity first, cavity last, surface micromachining, or any number of different fabrication methods. In at least one embodiment, a disclosed CMUT is manufactured using a thermal oxide growth on an SOI wafer. FIG. 1 illustrates a side view of a silicon-on-insulator (SOI) wafer 100. The SOI water may comprise a handle wafer 110, a buried oxide (BOX) layer 120, and a membrane layer 130. In one example embodiment, the fabrication of the CMUT may comprise the following parameters: 1.0 um+/−0.3 um, N++ Red Phosphorus Doping, 0.1 to 0.2 Ω/cm². The thermal oxide growth may comprise, for example, Å (angstroms) to 400 Å dry at 980° C.

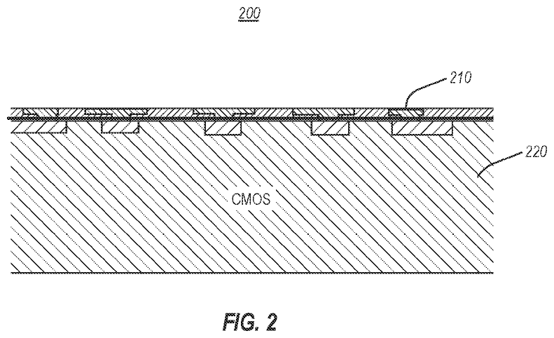

FIG. 2 illustrates a side view of a complementary metal-oxide semiconductor (CMOS) wafer 200 with lower electrodes 210 on a substrate 220. The substrate may be formed of silicon and may contain various circuitry such as, but not limited to, signal generation circuits and signal reception circuits. In at least one embodiment, the CMOS wafer 200 is fabricated using the following parameters: UTM M8 (3 um, Cu) on CMOS with 1.5 um oxide passivation with SiN as outgassing barrier. Via's may be added with a Cu damascene process, and the bottom electrode may be fabricated to 1000 A also using a Cu damascene process.

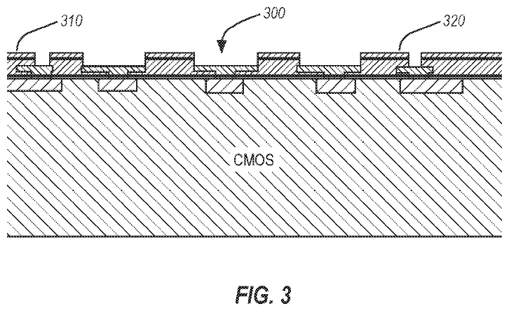

FIG. 3 illustrates a side view of a CMOS wafer with at least one cavity 300 formed in oxide layers. The CMOS wafer may now include a peripheral wall 310 defining the cavity 300 and one or more posts 320 positioned within the cavity 300. In one example embodiment, the CMOS wafer cavity may be constructed using the following parameters: 400 A CVD (chemical vapor deposition) oxide/450 A SiN, 6-8K HDP->CMP to SiN, peripheral wall and post etch to stop at SiOx, and HDP oxide 200 A.

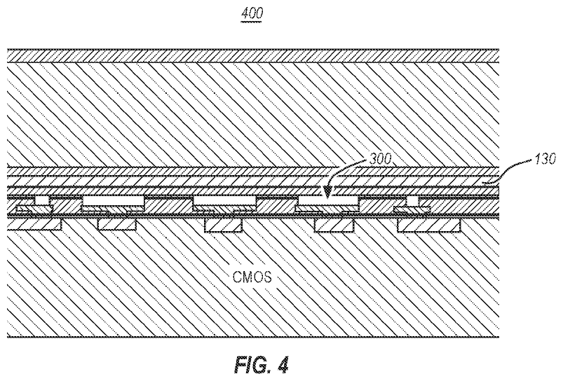

FIG. 4 illustrates a side view of a CMOS wafer bonding 400. In the depicted embodiment, the SOI wafer 100 of FIG. 1 is bonded to the CMOS wafer with at least one cavity 300 from FIG. 3. The membrane 130 is now positioned above the cavities. The fabrication may comprise a cleaning process such as an EKC® cleaning process, plasma activation and low temperature bonding, and post bonding annealing at 300° C. for 90 minutes. In at least one embodiment, the bonding temperature may be between approximately 400° and approximately 425° C. for half an hour or less. Additionally or alternatively, the bonding temperature may comprise a temperature of less than 400° C. The low temperature bonding results in the membrane 130 being bonded to peripheral wall 310 while, in at least some embodiments, not being bonded to one or more (or all) of the posts 320.

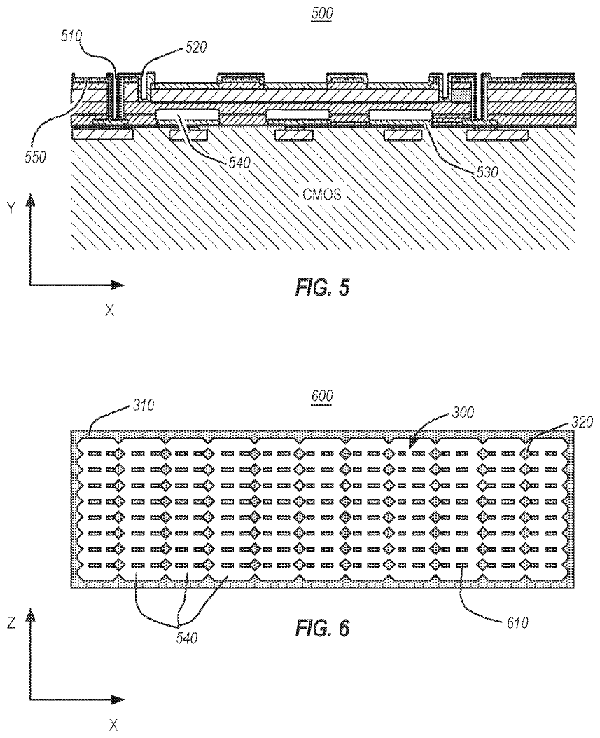

FIG. 5 illustrates a side view of a CMUT 500 after metallization and passivation. The resulting CMUT 500 now comprises one or more vias 510, one or more cells 540, and a trench etch 520 to isolate the conductive membrane 130 from the conductive circuitry integrated into the CMUT. The fabrication process may comprise a wafer grinding and etching process. The contacts 550 may be opened using an etch. Additionally, the fabrication process may comprise metal deposition for metal grid, Ti/1.5 um Al/Ti/1.5 um Al, BOX removal and a thinning etch on the membrane 130, and the trench etch. Further, fabrication may comprise a passivation layer deposition (e.g., with parameters of 0.5 um SiO2/0.5 um SiN) and a pad opening.

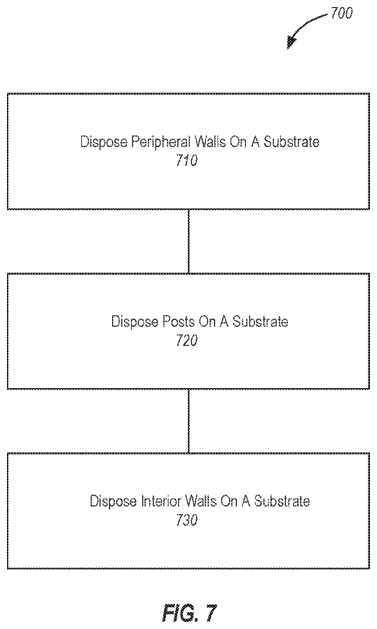

FIG. 6 illustrates a top view 600 of a CMOS wafter without a membrane. The depicted CMOS wafer comprises a top view of a peripheral wall 310 that circumscribes and defines the cavity 300, and one or more posts 320 positioned within the cavity 300. Also depicted are one or more interior walls 610 protruding from the substrate 220. The interior walls 610 are positioned within the boundaries of the peripheral walls 310 and may be located between adjacent posts 320. As depicted, in at least one embodiment, the posts 320 and interior walls 610 help to define individual cells 540, laid out in an array within the cavity 300. In the embodiment shown in FIGS. 5 and 6, the disclosed cavities exhibit a rectangular shape. Further, in at least one embodiment, the cells 540 are in fluid communication with each other such that a given cell 540 is not completely physically separated from its neighboring cell 540, but instead is fluidically open to its neighbors. The individual cells 540 function as discrete transducers with each cell having its own discrete and addressable electrode 530.

In at least one embodiment, the acoustic performance of the CMUT can be specified by controlling the location and sizes of the one or more posts 320 and the one or more interior walls 610. Additionally, the sizes and shapes of a given cell 540 or set of cells 540 need not be the same as its neighbors. As such, the cells 540 depicted in FIG. 6 may be shaped and sized differently such that they are tuned to a variety of different specific acoustic frequencies. Additionally, the relatively smaller sizes of the one or more posts 320 and the one or more interior walls 610 may allow for larger electrodes 530 (shown in FIG. 5), which can increase the sensitivity and performance of the CMUT.

Accordingly, the disclosed CMUT may comprise a substrate 220 with one or more peripheral walls 310 protruding from the substrate 220. As more clearly viewable in FIG. 6, the one or more peripheral walls 310 define an outer boundary of a cavity 300. A membrane 130 is bonded to the one or more peripheral walls 310. As depicted in FIG. 5, the cavity 300 is positioned between the membrane 130 and the substrate 220. One or more posts 320 protrude from the substrate 220. The one or more posts 320 are enclosed by the one or more peripheral walls 310.

As used herein, a “width” of an object is measured in the x-direction or z-direction as shown with respect to FIGS. 5 and 6 such that a width is a lateral measurement of a feature of the CMUT. In contrast, a “height” or “thickness” is measured in the y-direction as shown with respect to FIG. 5 such that the height or thickness is a vertical measurement of a feature of the CMUT. In particular, unless stated otherwise, a thickness or height is a vertical measurement of a feature starting from the cavity floor and extending to the top of the feature.

In at least one embodiment, the peripheral walls 310 comprise a width of at least 8 microns, at least 10 microns, or at least 12 microns. Additionally or alternatively, in at least one embodiment, at least a portion of the one or more posts 320 comprise a maximum cross-sectional width of less than 10 microns, less than 8 microns, or less than 5 microns. Further, in at least one embodiment, the interior walls 610 comprise a width (e.g., a lateral measurement) of less than 10 microns, less than 8 microns, or less than 5 microns.

During the bonding process in fabrication, a membrane 130 conventionally requires a minimum surface area in order to physically bond to a wall or post. In some embodiments, that minimum surface area may comprise a wall (e.g., the peripheral wall) with a width of at least 10 microns or a wall with a width of more than at least 8 microns. Similarly, in order to physically bond to a post during the bonding process in fabrication, the minimum area of a post may comprise a post having an upper surface area with a dimension of at least 10 microns (e.g., a square post measuring at least 10 microns by 10 microns or a post having a diameter of at least 10 microns) or a post having an upper surface area with a dimension of at least 8 microns (e.g., a square post measuring at least 8 microns by 8 microns or a post exhibiting a diameter of at least 8 microns). Accordingly, in some embodiments by creating one or more peripheral walls 310 having widths of at least 8 microns, and preferably at least 10 microns, the membrane is able to physically bond to the one or more peripheral walls 310. In contrast, in some embodiments by creating a one or more posts 320 having cross-sectional widths of less than 10 microns, and preferably less than 8 microns, the membrane remains physically unbonded to the one or more peripheral walls 310 during the fabrication bonding process. Similarly, in some embodiments by creating one or more interior walls 610 of less than 10 microns in width, and preferably less than 8 microns in width and more preferrable less than 1 micron, the membrane remains physically unbonded to the one or more interior walls 610 during the fabrication bonding process.

In at least one embodiment, the membrane 130 is unbonded to at least a portion of the one or more posts 320. Additionally or alternatively, in at least one embodiment, the membrane 130 is unbonded to at least a portion of the one or more interior walls 610. The one or more posts 320 and/or the one or more interior walls 610 may comprise heights that are, for example, 2 nanometers shorter (i.e., into the plane of the paper for the CMUT shown in FIG. 6) than the height of the peripheral walls 310. In other embodiments, the heights of one or more posts 320 and on or more interior walls 610 may be 1 micron shorter, 0.5 micron shorter, as little as 2 nanometers shorter than the height of the peripheral walls 310, or any range picking the aforementioned values as endpoints. Thus, the membrane 130 may be configured to bond with the peripheral walls 310 while remaining unbonded from at least a portion of the one or more posts 320 and the one or more interior walls 610. In some embodiments, the membrane 130 is unbonded to any of the one or more posts 320. In alternate embodiments, the membrane 130 is unbonded from a majority of the one or more posts 320.

In at least one embodiment, by utilizing one or more posts 320 and/or the one or more interior walls 610 of relatively smaller sizes (i.e., too small for physical bonding to the membrane) the resulting CMUT provides greater active area of the membrane 130. Additionally, the disclosed fabrication embodiment allows for more reliable CMUT production due to the relatively smaller area that requires physical bonding. In particular, since only the one or more peripheral walls 310 require bonding, there is a lower likelihood of manufacturing defects that can arise when compared to conventional bonding processes that may require bonding at the one or more peripheral walls 310, the one or more posts 320, and the one or more interior walls 610.

In at least one embodiment, the cavity 300 comprises a negative air pressure relative to outside the CMUT 500. During fabrication a vent hole may be drilled into the CMUT (e.g., through the peripheral wall 310). A vacuum may be applied to the vent hole causing a negative air pressure to develop within the cavity 300. The resulting pressure difference may cause the membrane 130 to be pressed down onto the one or more posts 320 and/or the one or more interior walls 610, causing the posts 320 and/or interior walls 610 to support or prop up the membrane 130 at their individual locations (e.g., like a “tent pole”). In at least one embodiment, creating cells 540 that are in fluid communication with each other also distributes initial internal pressure in the CMUT throughout the device. For example, in conventional CMUTs, a cavity at a corner of the device may comprise an internal pressure of up to 10 atm. This high pressure may cause the bonding of the CMUT to fail before the device can be depressurized. In contrast, the disclosed embodiments leave the cells 540 fluidically open to each other such that the pressure can spread through the device. This results in a lower per cell pressure, reduces the likelihood of the bonding failing prior to the apparatus being vented, and simplifies the degasification of the cavity 300.

One of skill in the art will appreciate that the use of “bonded” and “unbonded” herein refers to the status of the membrane 130 with respect to the one or more peripheral walls 310, the one or more posts 320, and the one or more interior walls 610 during fabrication. In practice, after fabrication, once a charge is applied to the membrane 130 and/or once the negative pressure within the cavity 300 pulls the membrane down onto the one or more posts 320 and the one or more interior walls 610, a bond, though relatively weak because of the available surface area, may ultimately form between the membrane 130 and at least a portion of the one or more posts 320 and the one or more interior walls 610.

The following discussion now refers to methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

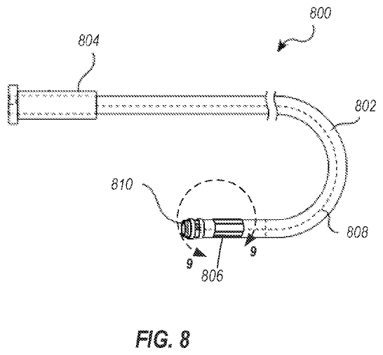

FIG. 7 depicts a flowchart of steps in a method 700 for fabricating a CMUT. The depicted method comprises an act 710 of disposing or forming peripheral walls 310 on a substrate. Act 710 comprises disposing or forming, on a substrate, one or more peripheral walls 310, the one or more peripheral walls 310 defining an outer boundary of a cavity, wherein the one or more peripheral walls 310 comprise a width of at least 8 microns (width being defined as noted above with respect to FIG. 6). The peripheral wall or walls may be formed using an additive or a subtractive process.

Method 700 may also include an act 720 of disposing or forming posts on a substrate. Act 720 comprises disposing, on the substrate, one or more posts, the one or more posts 320 laterally enclosed by the one or more peripheral walls 310, wherein at least a portion of the one or more posts 320 comprise a width of 1 micron to 10 microns. Additionally, method 700 may include an act 730 of disposing or forming interior walls 610 on a substrate. Act 730 comprises disposing or forming, on the substrate, one or more walls protruding from the substrate, the one or more walls laterally enclosed by the one or more peripheral walls, wherein at least a portion of the one or more walls comprise a width of 0.5 microns to 1 micron. The posts and/or walls may be formed using an additive or a subtractive process.

In additional embodiments, method 700 may further comprise bonding a membrane 130 to the one or more peripheral walls 310 with the cavity 300 being positioned between the membrane 130 and the substrate. Bonding the membrane 130 to the one or more peripheral walls 310 may be accomplished while leaving at least a portion of the one or more posts 320 and/or one or more interior walls 610 unbonded to the membrane 130. The method may further include outgassing or degasification of the cavity 300 to create a negative relative pressure within the cavity and to cause the membrane to contact at least some of the posts and/or interior walls.

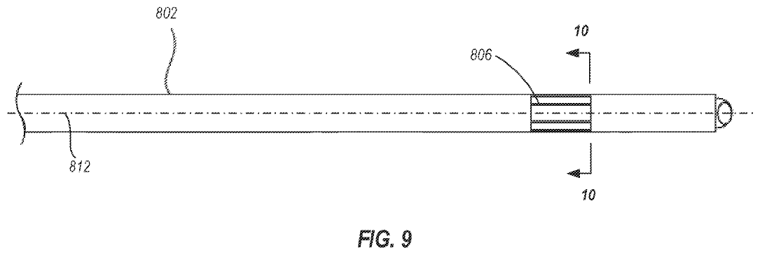

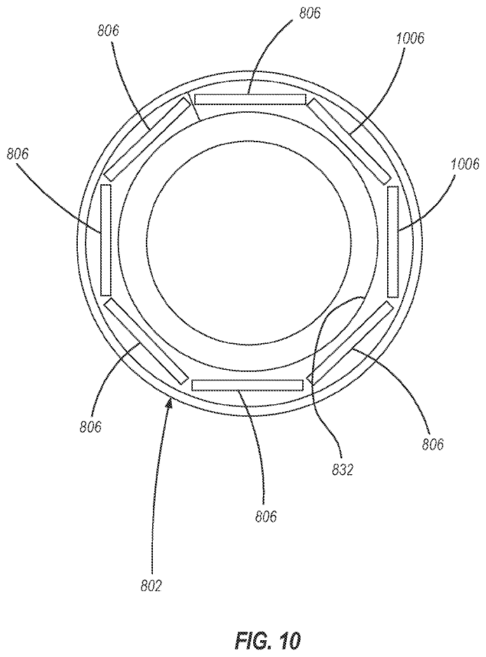

Referring to FIG. 8, a catheter 800 is shown. While a catheter is presented as an example in association with FIG. 8 (and related FIGS. 9-11), components, features and embodiments described can also be applied to constructions of guidewires or other elongated. flexible elements. The catheter 800 includes an elongated, flexible body 802 (also referred to herein as an “elongated, flexible member”), and may be associated with a proximal device 804. In some embodiments, the proximal device 804 may include a control unit (not shown) and/or a valve device such as described, for example, in U.S. Pat. No. 11,304,659 issued on Apr. 19, 2022, and entitled OPERATIVELY COUPLED DATA AND POWER TRANSFER DEVICE FOR MEDICAL GUIDEWIRES AND CATHETERS WITH SENSORS, or U.S. patent application Ser. No. 17/979,629 filed on Nov. 2, 2022, and entitled DATA AND POWER TRANSFER DEVICES FOR USE WITH MEDICAL DEVICES AND RELATED METHODS, the disclosures of which are incorporated by reference herein in their entireties.

The catheter 800 includes one or more sensors 806. Power wires and/or data lines 808 may extend along the length of the catheter 800 to the one or more sensors 806 near a distal end 810 of the elongated body 802. As used herein, a “power line” and/or “data line” refer to any electrically conductive pathway (e.g., traces) within or on the medical device. Although multiple power and/or data lines may be utilized, some embodiments may be configured to send both power and data on a single line and/or manage sensor data signals from multiple sensors on a single line. This reduces the number of lines that must be routed through the structure of the catheter 800 and more effectively utilizes the limited space of the device, as well as reducing the complexity of the device and the associated risk of device failure.

The proximal device 804 may include one or more ports to facilitate the introduction of fluids (e.g., medications, nutrients, nanoparticle colloidal solutions) into the catheter 800. The body 882, or at least a distal portion thereof, may be sized and configured to be temporarily inserted in the body and configured, for example, to provide diagnostic information or to deliver an implant in the body. In one embodiment, the catheter 800 is a peripherally inserted central catheter (PICC) line, typically placed in the arm or leg of the body to access the vascular system of the body. The catheter 800 may also be a microcatheter, a central venous catheter, an IV catheter, coronary catheter, stent delivery catheter, balloon catheter, atherectomy type catheter, or IVUS catheter or other imaging catheter. The catheter 800 may be a single or multi-lumen catheter.

Referring to FIG. 9, one or more sensors 806 of the catheter 800 may include, for example, a pressure sensor, a flow sensor, an imaging sensor, a component detection sensor, or combinations thereof, for example. Additionally, while generally referred to as a “sensor” in discussing various embodiments throughout, such “sensor” components (e.g., sensor 906) may comprise or otherwise be associated with transducers or other components and may be configured as input devices, output devices, or both.

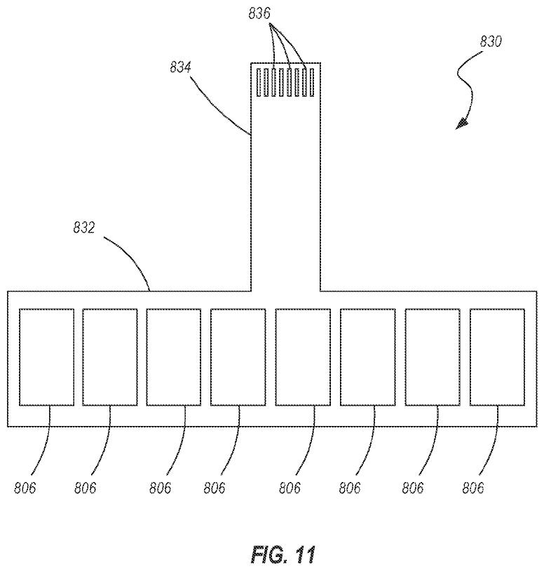

The sensors 806, as depicted in FIGS. 9 and 10, are arranged circumferentially about a longitudinal axis 812 of the elongated body 802. In the embodiment shown in FIG. 9, the sensors 806 include ultrasonic transducers that may be used for imaging (e.g., imaging of a vessel), for activating or releasing a therapeutic, or for some other purpose. In one embodiment, the ultrasonic transducers may include a capacitive micro-machined ultrasonic transducer (CMUT), such as described hereinabove. In other embodiments, the ultrasonic transducers may include piezoelectric transducers, including piezoelectric micromachined ultrasonic transducers (PMUTs), or some other type of ultrasonic device.

While depicted in FIGS. 9 and 10 to include eight separate transducers/sensors 806 circumferentially disposed about a longitudinal axis 812 of the body 802, other numbers of sensors 806 may be used and other geometric and spatial arrangements may be utilized. The catheter 800, as depicted in FIGS. 1-3 may, thus, be utilized as a side-looking intravenous ultrasound (IVUS) catheter to image a vessel (e.g., a coronary vessel) in determining, for example, whether stenosis or some other condition has occurred and whether some particular intervention may be warranted.

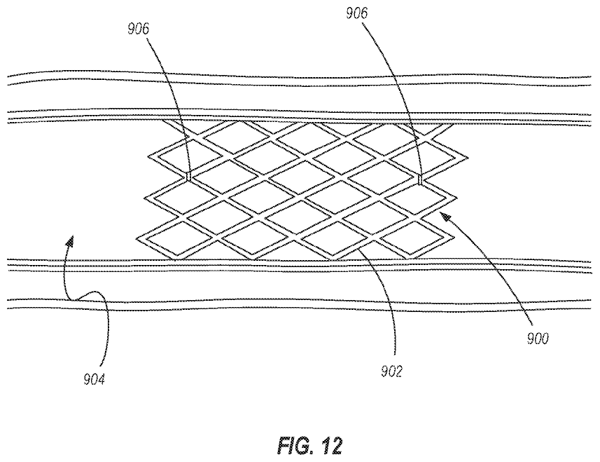

Referring briefly to FIG. 11, a CMUT array 830 is shown which may be used to form the array of circumferentially disposed sensors 806 in a catheter 800 as depicted in FIGS. 9 and 10, or in some other device where an array CMUTs or other sensors are desired. The array 830 includes a flexible substrate 832 and a plurality of sensors/CMUTs 806. In one embodiment, the flexible substrate may include a polyimide material. In one embodiment, the sensors/CMUTs 806 may be disposed on the flexible substrate 832, in other embodiments, the sensors/CMUTs 806 may be sandwiched between multiple layers of a flexible material or otherwise encased by a flexible material. A flexible tail 834, which may be integrally formed with the flexible substrate 832, may extend from the sensors/CMUTs 806 and provide a plurality of connection pads 836 for connection with other electronics (e.g., another flexible circuit, ribbon cable, individual conductors, etc.). Circuitry may be formed within the substrate 832/tail 834 to connect the individual sensors/CMUTs 806 together, to connect the sensors/CMUTs 806 with the connection pads 836, and/or to connect other electronic components associated with the CMUT array 830.

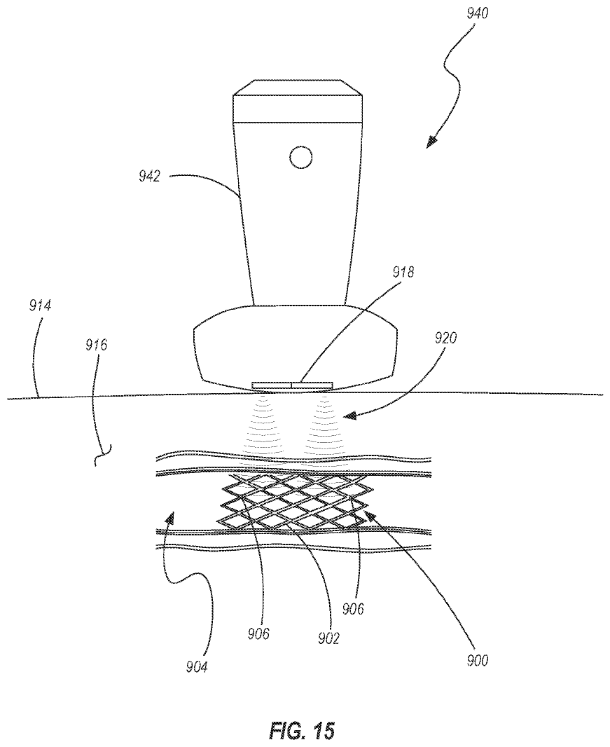

When implemented into a catheter or other elongated body, the CMUT array 830 may be “rolled” into a configuration such as shown in FIGS. 9 and 10, and the connection pads 836 may be coupled with power/data lines (e.g., line 808 shown in FIG. 15). Again, while shown to include a single row of eight sensors/CMUTs 806, the CMUT array 830 may exhibit other configurations, including different quantities of sensors/CMUT 806, multiple rows of sensors/CMUTs, staggered spatial arrangements of sensors/CMUTs 806 and the like. Additionally, in some embodiments, the CMUT array 830 may include CMUTs of different specifications, including at least two CMUTs configured to operate at different frequencies.

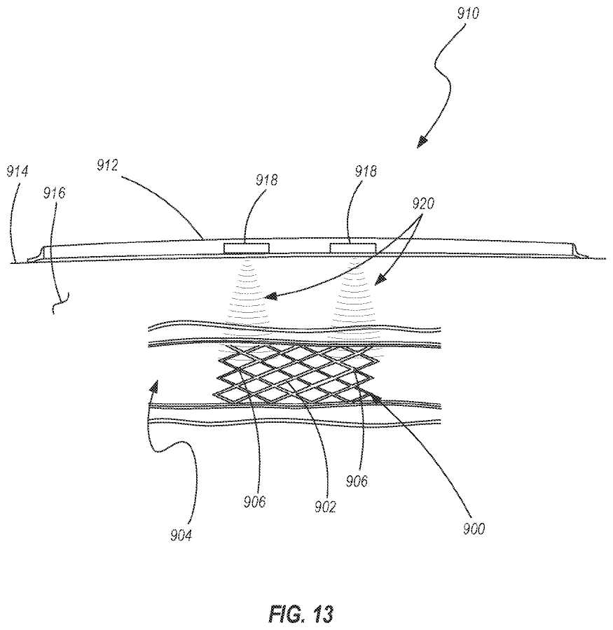

Referring to FIG. 12, a medical device in the form of a stent 900 is shown according to an embodiment of the present disclosure. The stent may include a body 902 positioned within a vessel 904 to act as a scaffolding or a support structure at a desired location within a vessel 904 (e.g., within a calcified portion of a coronary artery). As will be appreciated by those of ordinary skill in the art, the stent 900 may be delivered by way of a catheter in an initially radially collapsed state, and then be radially expanded at a desired location to expand the vessel and open the lumen of the vessel for increased fluid flow. One or more electronic components 906 (e.g., sensors such as described hereinabove, systems on a chip (SOC)) may be coupled to, embedded within, or otherwise associated with the stent 900. For example, in one embodiment, the electronic components may include an ultrasonic transducer (e.g., a CMUT chip such as discussed above) along with some other type of sensor (e.g., a pressure sensor, a flow sensor, a proximity sensor which may be separate from, or integrated into, a CMUT chip). The electronic components 906 may be “passive” in the sense that they are not powered or actively sensing or processing until affirmatively activated by a specific, external stimuli. In the embodiment shown in FIG. 12, there are two separate electronic components 906, with one disposed near each longitudinal end of the stent 900 (e.g., one distally located and one proximally located).

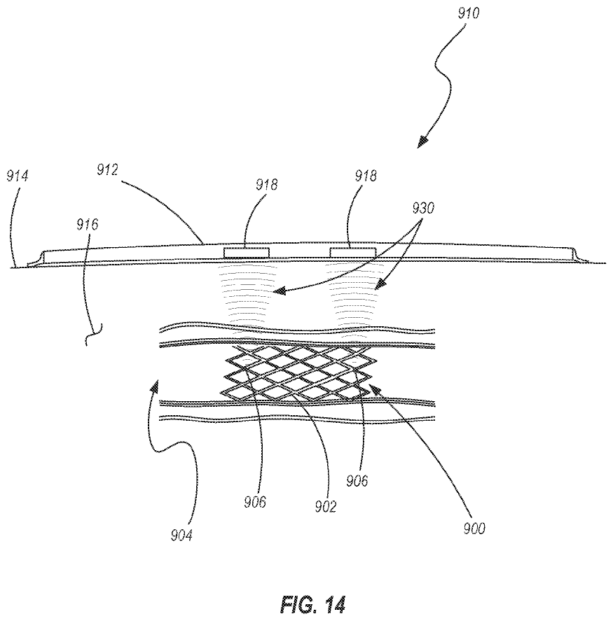

As seen in FIGS. 6 and 7, the stent 900 may be used as part of a system 910, wherein a patch or a pad 912 is placed on an exterior surface 914 of the tissue 916 of a user. As will be discussed in further detail below, the patch 912 may include one or more ultrasonic transducers 918 configured to emit ultrasonic waves 920 (also referred to as acoustic waves) at one or more desired frequencies into the tissue 916 and to the electronic components 906. In some cases, the waves 822 may be focused, such as by beam forming or using other known techniques so that energy from the acoustic waves 920 is focused on a desired location (e.g., at the location of the electronic components 906). As shown in FIG. 13, ultrasonic waves may be transmitted to the electronic components 906 and the energy from the acoustic waves 920 may be used to power the electronic components 906 through reception of the ultrasonic waves by ultrasonic transducers (e.g., CMUTs) associated with the electronic components 906.

When the electronic components 906 are powered, they may operate according to a desired protocol to perform desired functions. For example, the electronic components 906 may include a sensor to detect a parameter associated with the health and function of the vessel 904 and or the condition or state of the stent 900. In one embodiment, the electronic components 906 may include pressure sensors wherein fluid pressure may be measured to determine whether there is significant blockage within the vessel at the location of the stent 900 (e.g., recalcification). For example, pressure may be measured at each end of the stent to determine if a pressure drop has occurred beyond a determined threshold value. In the case that a pressure drop has occurred beyond an acceptable value, intervention may be required. However, determination of whether intervention is required may be, at least preliminarily, determined without additional invasive techniques. In other embodiments, other parameters may be determined by the sensors, such as flow rates, temperature, or other information relevant to diagnosis of the health of the vessel.

Referring to FIG. 14, ultrasonic signals (referred to herein as “response signals 930” to differentiate from the originally transmitted ultrasonic signals 920) may be transmitted from the electronic components 906 to the patch 912 after the associated sensors have determined their specified parameters or characteristics. This may occur in a variety of different ways. In one example, the electronic components 906 may process the information obtained by any associated sensors and then transmit the response signals 930 by associated transducers (e.g., CMUTs) back to the patch 912.

In another embodiment, when a sensor associated with an electronic component 906 detects a particular parameter state (e.g., a pressure above a specified threshold), a membrane, disc, magnetic device, or some other component may be altered, causing the transmitted acoustic waves 920 (FIG. 13) to be reflected in a specified way, creating the response wave 930. The system associated with the patch 910 recognizes this response signal to indicate a particular parameter state has been detected. Such a system requires relatively little power, enabling the electronic components to be significantly reduced in size.

Still referring to FIGS. 6 and 7, in another embodiment, the electronic components 906 may include proximity sensors, or the system 910 may include additional processors or sensors to determine the relative location of each (or a select subset) of the electronic components 906. Thus, for example, based on the relative locations of two or more electronic components 906, it may be determined whether the body 902 of a stent 902 has been sufficiently expanded (e.g., as a stent body 902 expands radially, it may also contract longitudinally—or shorten—changing the positions of the electronic components 906 relative to each other).

The electronic components 906 (in the above example as well as in other embodiments) may include sensors to detect a variety of other parameters or characteristics associated with the health of an individual or the performance of a medical device implanted within the individual. For example, the electronic components 906 may include sensors that are configured to sense the presence of biological components or measure physiological parameters in a targeted anatomical location. Example biological components and physiological parameters that may be detected/measured include sugar levels, pH levels, CO2 levels (CO2 partial pressure, bicarbonate levels), oxygen levels (oxygen partial pressure, oxygen saturation), pressure, temperature, and other such substrates and physiological parameters. The one or more sensors may be configured to sense the presence, absence, or levels of biological components such as, for example, immune system-related molecules (e.g., macrophages, lymphocytes, T cells, natural killer cells, monocytes, other white blood cells, etc.), inflammatory markers (e.g., C-reactive protein, procalcitonin, amyloid A, cytokines, alpha-1-acid glycoprotein, ceruloplasmin, hepcidin, haptoglobin, etc.), platelets, hemoglobin, ammonia, creatinine, bilirubin, homocysteine, albumin, lactate, pyruvate, ketone bodies, ion and/or nutrient levels (e.g., glucose, urea, chloride, sodium, potassium, calcium, iron/ferritin, copper, zinc, magnesium, vitamins, etc.), hormones (e.g., estradiol, follicle-stimulating hormone, aldosterone, progesterone, luteinizing hormone, testosterone, thyroxine, thyrotropin, parathyroid hormone, insulin, glucagon, cortisol, prolactin, etc.), enzymes (e.g., amylase, lactate dehydrogenase, lipase, creatine kinase), lipids (e.g., triglycerides, HDL cholesterol, LDL cholesterol), tumor markers (e.g., alpha fetoprotein, beta human chorionic gonadotrophin, carcinoembryonic antigen, prostate specific antigen, calcitonin), toxins (e.g., lead, ethanol), and/or white blood cell count or red blood cell count (e.g., using a microfluidic chamber). Additionally, or alternatively, sensors may be used to detect evidence of thrombosis, the impedance of a tissue structure (or across an implanted medical device), water content (e.g., to detect the change in density or size of a tumor), or the physical size of a desired anatomical structure (e.g., based on the proximity of implanted electronic components).

Referring briefly to FIG. 15, another system 940 is shown in accordance with another embodiment of the present disclosure. The system 940 includes a stent 900 with electronic components 906 as previously described, but rather than using a patch adhered to, or otherwise placed on a patient's skin, a handheld (e.g., portable) ultrasound probe 942 is employed. The probe 942 includes one or more ultrasonic transducers 918 to provide the energy for powering and/or communicating with the electronic components 906 in a manner such as described above. The probe 940 may be associated with other systems and hardware or may be a stand-alone device.

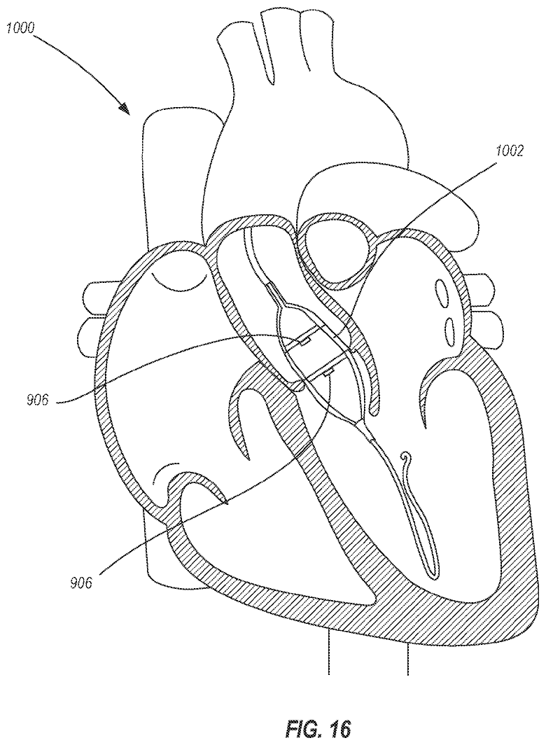

Referring briefly to FIG. 16, a human heart 1000 is illustrated while a transaortic valve replacement (TAVR) procedure is underway, wherein a new valve 1002 is placed within the natural, diseased aortic valve. The new valve 1002 may include one or more electronic components 906 such as described above with respect to the stent embodiment. The electronic components 906 may be used as part of a system (e.g., using a patch or ultrasonic probe) such as described hereinabove. The electronic components 906 may be used, for example, to measure pressure or flow across the new valve 1002, and/or to measure proximity of the electronic components 906 to determine whether the new valve has properly expanded, whether it is functioning as intended, to determine flow rate through the valve, or to determine some other parameter, characteristic or state associated with the functioning of the valve and the heart.

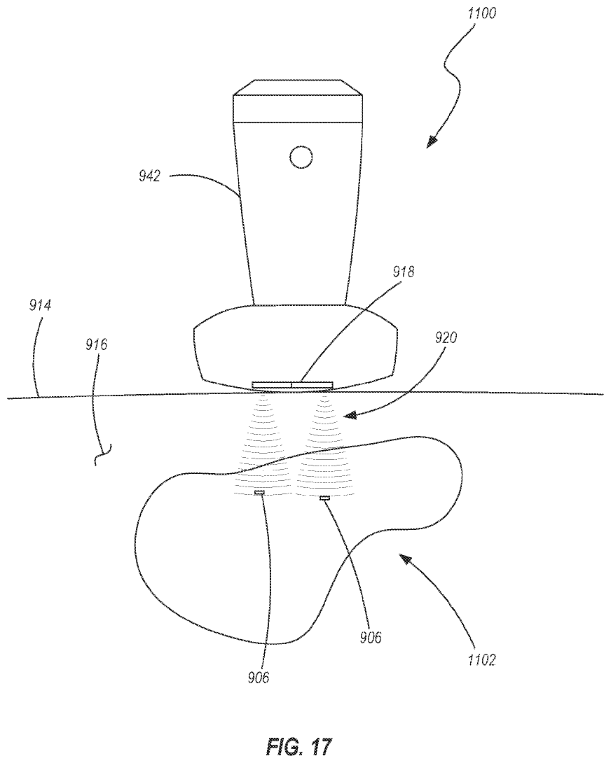

Referring to FIG. 17, another system 1100 is shown according to another embodiment of the present disclosure. The system 1100 includes electronic components 906 embedded or implanted within a tumor 1102. The electronic components 906 may be implanted within the tumor in association with another procedure or may be implanted during a specific procedure. For example, the electronic components 906 may be delivered to the tumor 1102 via a needle. While the electronic components 906 are shown being within the mass of the tumor 1102, in some embodiments they may be implanted to be immediately adjacent to the tumor or they may be strategically located at another location in association with the tumor's impact on the function of an organ or anatomical structure.

The electronic components 906 be configured such as previously described, being powered by ultrasonic waves 920 and having sensors to determine a parameter, characteristic, or state relating to the tumor 1102. A probe 942 includes one or more ultrasonic transducers 918 to provide the energy for powering and/or communicating with the electronic components 906 in a manner such as described above. In other embodiments, a patch or pad may be used such as previously described. In one embodiment, the electronic components 906 may include a sensor configured to detect water content of the tumor 1102. Upon sensing the water content of the tumor 1102, information may be relayed back to the probe 942 (or to some other device) as described hereinabove with respect to other embodiments. The information provided by the electronic components 906 may be used in determining the density of the tumor 1102, thereby ascertaining whether the tumor 1102 has shrunk, grown, or is relatively unchanged from a prior determination of the tumor's size. The system 1100, therefore, enables frequent monitoring of a tumor being treated (e.g., by radiation, chemotherapy, or some other approach), to make an early determination of the effectiveness of the treatment. In many cases, a prescribed course of treatment indicates its effectiveness early during the process. However, conventional means of monitoring typically require the use of imaging modalities that are expensive and that expose the patient to harmful radiation. Thus, such imaging usually is not done until the patient has been subjected to the treatment for an extended period of time (e.g., 6-8 weeks). Early monitoring of the tumor's response to a course of treatment using the described system (e.g., within a week or two of initiating treatment) enables a physician to determine early whether course corrections need to be made, saving valuable time and resources.

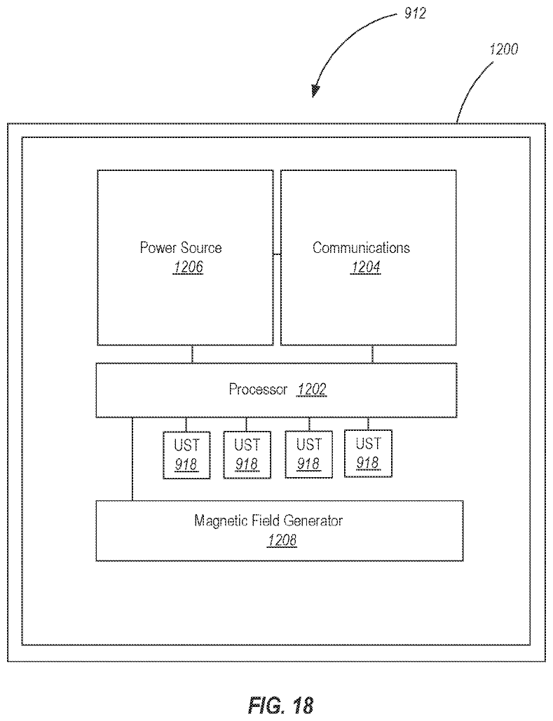

Referring now to FIG. 18, a schematic is shown of an example patch 912 having ultrasonic transducers 918. The patch 912 may include a flexible film 1200 (e.g., a polyimide or other polymer material) which may be adhered to a patient's skin. Various components may be disposed on or in the film 1200. For example, the patch 912 may include one or more ultrasonic transducers 918 of one or more designs. In one embodiment, the ultrasonic transducers 918 may include a capacitive transducer such as a CMUT (capacitive micromachined ultrasonic transducer). In other embodiments, the transducer may include a piezoelectric transducer such as a PMUT (piezoelectric micromachined ultrasonic transducer). In some embodiments, piezoelectric ultrasound transducers may be formed using techniques such as described hereinabove. The ultrasonic transducers 918 may include individual transducers or arrays of ultrasonic transducers. Additionally, the transducers 918 may be configured to operate on multiple frequencies. Thus, for example, one frequency may be used to release the power the electronic components 906, while another frequency may be used to image an area of interest within an individual's anatomy.

The patch 912 may additionally include a processor 1202 such as an ASIC (application specific integrated circuit) or other appropriate integrated circuit. A communication device 1204 (e.g., an integrated circuit) may be used to provide wireless communication, such as via Bluetooth, Wi-Fi, Zigbee, or some other protocol, between the patch 912 and external devices such as a personal computer, a tablet computer, a smartphone, or other such device. The external device may be used to control the patch, and or to receive information from the patch, such as time and/or duration of activation. In some embodiments, a dedicated button or switch may be provided with the patch 912 to activate the patch 912 (e.g., activate the ultrasonic transducers 918 of the patch 912), instead of, or in addition to, the patch being activated or controlled by an external device. A power source 1206 such as a battery, a capacitor, or a connector to an external power source, may also be included to provide power to the various components of the patch.

While the example shown in FIG. 18 depicts discrete components such as one or more transducers 918 and one or more processors 1202, such components may be integrated. For example, a CMUT on CMOS (complementary metal-oxide semiconductor) or PMUT on CMOS may be used in place of individual transducers 918 and processors 1202.

Still referring to FIG. 18, in some embodiments, a magnetic field generator 1208 (e.g., an electromagnet) may be used to assist focusing, steering, or otherwise manipulating the ultrasonic waves (the emitted waves, the response waves, or both). In some instances, the magnetic field may be used to influence the disc, membrane or other structure used to influence backscattered response waves.

Various embodiments herein have been provided by way of example. However, other implementations and configurations are also contemplated. For example, an electronic component (e.g., 906) may be associated with a left atrial appendage implant to help monitor issues relating to AFIB such as pressure or potential thrombosis development. In other embodiments, electronic components may be implanted in organs (e.g., the heart), either directly or in combination with some other medical device, to monitor a desired parameter such as elasticity, density, or the like. In other embodiments, electronic components may be implanted to monitor or detect transplant rejection, whether such be cell mediated or antibody mediated rejection. In other embodiments, the electronic component may include an electrode to provide current or voltage a desired location within the anatomy of the patient when powered and instructed by an external device (e.g., a patch or probe). In other embodiments, the electronic component may include a therapeutic agent that is released at a targeted location when powered an instructed by an external device.

The various embodiments described above may find particular relevance in high-frequency ultrasound applications—for example frequencies of 20 MHz and above. For example, applications such as intravascular ultrasound (IVUS) imaging may benefit from such embodiments. In IVUS, the overall size of the transducer or the transducer array may be limited to 1 mm or smaller in any given direction. When considering a device incorporating an array of transducers operating in the 20 MHz-60 MHz range, the size of each array element would be about 50 μm (microns) down to about 10 μm. In these cases, the area of non-moving regions of the CMUT structure effectively reduces the active area and can be severely limiting, even when micromachining techniques are used. For example, 8 μm to 10 μm wide walls separating the CMT membranes, such as is used in conventional wafer bonding processes, can reduce the active area significantly. The walls and posts described herein help to increase, if not maximize, the transmitting and receiving performance of the device at desired operational frequencies. Embodiments of the present disclosure help increase performance of CMUT devices while maintaining acceptable yield and reliability of the resulting devices.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims.

1. A capacitive micromachined ultrasonic transducer (CMUT) apparatus comprising:
a substrate;
one or more peripheral walls protruding from the substrate, the one or more peripheral walls defining an outer boundary of a cavity;
a membrane bonded to the one or more peripheral walls, the cavity being positioned between the membrane and the substrate;
one or more posts protruding from the substrate, the one or more posts enclosed by the one or more peripheral walls, wherein at least a portion of the one or more posts comprise a width of less than 10 microns; and
one or more interior walls protruding from the substrate, the one or more interior walls enclosed by the one or more peripheral walls.

2. The CMUT apparatus of claim 1, wherein the one or more peripheral walls comprise a width of at least 8 microns.

3. The CMUT apparatus of claim 1, wherein the membrane is unbonded to at least a portion of the one or more posts.

4. The CMUT apparatus of claim 3, wherein the membrane is unbonded to at least a portion of the one or more interior walls.

5. The CMUT apparatus of claim 4, wherein the membrane is unbonded to any of the one or more posts.

6. The CMUT apparatus of claim 1, wherein the cavity comprises a negative air pressure relative to outside the CMUT apparatus.

7. The CMUT apparatus of claim 1, wherein the one or more posts are at least 2 microns shorter than the one or more peripheral walls.

8. The CMUT apparatus of claim 1, wherein the one or more posts, wherein at least a portion of the one or more interior walls comprise a width of less than 1 micron.

9. A method for constructing a capacitive micromachined ultrasonic transducer (CMUT) apparatus comprising:
disposing, on a substrate, one or more peripheral walls, the one or more peripheral walls defining an outer boundary of a cavity, wherein the one or more peripheral walls comprise a width of at least 8 microns;
disposing, on the substrate, one or more posts, the one or more posts enclosed by the one or more peripheral walls, wherein at least a portion of the one or more posts comprise a width of 1 micron to 10 microns; and
disposing, on the substrate, one or more interior walls protruding from the substrate, the one or more interior walls enclosed by the one or more peripheral walls, wherein at least a portion of the one or more interior walls comprise a width of 0.5 microns to 1 micron.

10. The method of claim 9, further comprising:
bonding a membrane to the one or more peripheral walls, the cavity being positioned between the membrane and the substrate.

11. The method of claim 10, wherein bonding a membrane to the one or more peripheral walls is accomplished while leaving at least a portion of the one or more posts unbonded to the membrane.

12. The method of claim 10, wherein the membrane is unbonded to at least a portion of the one or more interior walls.

13. The method of claim 10, wherein the membrane is unbonded to any of the one or more posts.

14. The method of claim 9, further comprising removing air from the cavity such that a negative air pressure relative to outside the CMUT apparatus is created within the cavity.

15. The method of claim 9, further comprising configuring the one or more posts to be at least 2 nanometers shorter than the one or more peripheral walls.

16. A medical device comprising:
a body;
at least one electronic component associated with the body, the at least one electronic component including:
at least one sensor, and
at least one ultrasonic transducer configured to receive ultrasonic signals and thereby power the at least one sensor,
wherein the at least one electronic component is configured to provide a response ultrasonic wave responsive to a determination by the at least one sensor.

17. The medical device of claim 16, wherein the at least one ultrasonic transducer includes a capacitive micromachined ultrasonic transducer (CMUT).

18. The medical device of claim 16, wherein the at least one ultrasonic transducer includes a capacitive micromachined ultrasonic transducer on complementary metal oxide silicon (CMUT on CMOS) device.

19. The medical device of claim 18, wherein the at least one sensor includes at least one of a pressure sensor and a flow sensor.

20. The medical device of claim 18, wherein the body includes a stent.

21. A system comprising:
an external device having a first ultrasonic transducer;
a medical device configured to be positioned within a patient's anatomy, the medical device comprising:
at least one electronic component:
at least one sensor, and
a second ultrasonic transducer configured to receive ultrasonic signals generated by the first ultrasonic transducer to power the at least one sensor,
wherein the at least one electronic component is configured to provide a response ultrasonic wave to the external device responsive to a determination by the at least one sensor.

22. The system of claim 21, wherein at least one of the first ultrasonic transducer and the second ultrasonic transducer includes a capacitive micromachined ultrasonic transducer (CMUT).

23. The system of claim 22, wherein the at least one of the first ultrasonic transducer and the second ultrasonic transducer includes a capacitive micromachined ultrasonic transducer on complementary metal oxide silicon (CMUT on CMOS) device.

24. The system of claim 21, wherein the at least one sensor includes at least one of a pressure sensor and a flow sensor.

25. The system of claim 21, wherein the at least one sensor is configured to detect at least one of a sugar level, a pH levels, a CO2 level, an oxygen level, a pressure, and a temperature.

26. The system of claim 21, wherein the at least one sensor is configured to detect a water content of a tumor.

27. The system of claim 21, wherein the at least one electronic component is coupled with a stent.

28. The system of claim 21, wherein the at least one electronic component is coupled with an aortic valve.

29. The system of claim 21, wherein the external device includes a patch configured for adherence to a patient's skin.

30. The system of claim 21, wherein the external device includes a handheld probe.

31. The system of claim 21, wherein the external device includes a magnetic field generator.

32. The system of claim 21, wherein the external device includes a power source, a processor, and a wireless communications device.

33. A method comprising:
implanting an electronic component within a patient adjacent to, or within, a tumor;
powering the electronic component using ultrasonic energy;
detecting a water content of the tumor using a sensor of the electronic component;
providing a response signal from the electronic component to an external device based on the detected water content; and
determining a density of, or a change in density of, the tumor based on the response signal.

34. A medical device comprising:
a flexible, elongated member having a distal portion configured for insertion into a vessel of a patient;
an imaging device disposed in the distal portion of the flexible, elongated member, the imaging device comprising:
a flexible substrate;
a plurality of ultrasonic transducers arranged on the flexible substrate, each of the plurality of ultrasonic transducers comprising a capacitive micromachined ultrasonic transducer on complementary metal oxide silicon (CMUT on CMOS) device; and
a tail extending from the flexible substrate having a plurality of connection pads.

35. The medical device of claim 34, wherein the plurality of ultrasonic transducers are circumferentially arranged about a longitudinal axis of the flexible, elongated member.
